(19) United States
(12) Patent Application Publication
(10) Pub. No.: US 2023/0190776 A1
(43) Pub. Date: Jun. 22, 2023

(54) ENCAPSULATION OF BIOACTIVE INGREDIENTS BY MULTIPLEX EMULSION

(71) Applicant: The Board of Regents of the University of Texas System, Austin, TX (US)

(72) Inventors: Karen Lozano, McAllen, TX (US); Victoria Padilla, Edinburg, TX (US); Acharan S. Narula, Chapel Hill, NC (US)

(21) Appl. No.: 18/069,796

(22) Filed: Dec. 21, 2022

Related U.S. Application Data
(60) Provisional application No. 63/292,400, filed on Dec. 21, 2021.

(51) Int. Cl. A61K 31/7016; A61K 47/36; A61K 31/222; A61K 31/4745; A61K 47/34; D04H 3/009; D04H 3/011; D01F 1/10; D01F 6/62; D01F 9/00

(52) U.S. Cl. CPC A61K 31/7016; A61K 31/222; A61K 31/4745; A61K 47/34; A61K 47/36; D01F 1/10; D01F 6/625; D01F 9/00; D04H 3/009; D04H 3/011; D10B 2509/00

(57) ABSTRACT

Described herein are various three-dimensional fiber structures that have multiple polymer fiber layers with an active therapeutic agent entrained in the polymer fiber layers. Further described are methods for forming the three-dimensional fiber structures where the method includes centrifugal spinning of an emulsion containing polymer(s) and the active therapeutic agent(s).

### PRIORITY CLAIM

The present application claims priority to U.S. Provisional App. No. 63/292,400, entitled “ENCAPSULATION OF BIOACTIVE INGREDIENTS BY MULTIPLEX EMULSION” filed Dec. 21, 2021, the disclosure of which is incorporated by reference herein in its entirety.

### BACKGROUND OF THE INVENTION

### 1. Field of the Invention

The disclosed embodiments generally relate to the field of fiber structures. Specific embodiments relate to the production of three-dimensional fiber structures that confine bioactive materials in the fiber layers of the structures.

### 2. Description of the Relevant Art

Globally, the healthcare system spends tens of billions of dollars per year on the treatment of wounds. Recently, nanofibers systems have entered the healthcare market providing promising results for alleviating and expediting healing. Nanofibers, for instance, have been sold as prepackaged nanofiber based (2D) gauzes. In addition to the wound care industry, benefits of nanofiber systems may also be found in the cosmetic industry, drug delivery, filtration, battery electrodes, and more.

Nanofibrous materials and nanofiber (NFs) systems have proven effective to heal wounds, such as both chronic and acute wounds. Chronic wounds include burn, ulcers associated with ischemia, diabetes Mellitus (i.e. diabetic foot, leg), venous stasis disease. Acute wounds, on the other hand, are related to those that usually heal on its own, depending on the extent, size, and depth of injury in the dermis and epidermis lining of the skin. Nanofibers possess high surface area to volume ratio, which provides a platform to mimic the extracellular matrix and enhance cellular growth and tissue regeneration. Cell adhesion, motion, and gene expression are also promoted. NFs provide a barrier to both mechanical trauma and pathogens, allow gaseous/fluid exchange, absorb exorbitant amounts of exudate, and avoid adherence to the wound during removal (or alternatively could become part of the body during healing). In addition, NFs can be embedded with a variety of other substances including painkillers and enzymes to alleviate pain and/or support the healing process.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1A illustrates the chemical structure of Salvianolic acid A.

FIG. 1B illustrates the chemical structure of Salvianolic acid B.

FIG. 2 illustrates the chemical structure of Oleanolic acid.

FIG. 3 illustrates the chemical structure of Trehalose.

FIG. 4 illustrates the chemical structure of Oxymatrine.

FIG. 5 illustrates the chemical structure of matrine.

FIG. 6 illustrates the chemical structure of Baicalin.

FIG. 7 illustrates the chemical structure of Poloxamer.

FIG. 8 illustrates the chemical structure of Eudragit®.

FIG. 9 depicts an example multiplex emulsion, according to some embodiments.

FIG. 10 depicts example electron microscope scans of types of 3D multifunctional nanofiber structures obtained using the procedures described herein, according to some embodiments.

FIG. 11 is a flow diagram illustrating a method for forming a three-dimensional fiber structure, according to some embodiments.

FIG. 12 is a flow diagram illustrating another method for forming a three-dimensional fiber structure, according to some embodiments.

Although the embodiments disclosed herein are susceptible to various modifications and alternative forms, specific embodiments are shown by way of example in the drawings and are described herein in detail. It should be understood, however, that drawings and detailed description thereto are not intended to limit the scope of the claims to the particular forms disclosed. On the contrary, this application is intended to cover all modifications, equivalents and alternatives falling within the spirit and scope of the disclosure of the present application as defined by the appended claims.

### DETAILED DESCRIPTION OF THE PREFERRED EMBODIMENTS

It is to be understood the present disclosed embodiments are not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The examples set forth herein are included to demonstrate preferred embodiments. It should be appreciated by those of skill in the art that the techniques disclosed in the examples which follow represent techniques discovered by the present inventors to function well in the practice of the disclosed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosed embodiments.

Nanofibers (NFs) have shown promising results in the four sequential phases of wound healing: hemostasis, inflammatory, proliferative, and remodeling phases. In the first phase of hemostasis, platelet and a coagulation cascade are activated to avoid blood loss and to promote platelet aggregation and thrombus formation. Through the platelet activation, a large number of growth and coagulation factors are released to help in the formation of blood clots. In the inflammatory phase, a vasodilation occurs allowing the release of histamine and other vasoactive mediators. Neutrophils begin to be attracted to the vascular endothelial cells of the wound by a variety of chemotactic substances. Neutrophils are responsible for phagocytosis, destruction of bacterial structures and removal of residual corpses and damaged tissues. In the proliferative phase, the fibroblasts cells migrate to the wound to activate and deposit an extra cellular matrix (ECM) that includes, for example, collagen, fibrin, fibronectin, and hyaluronic acid. As the proliferative phase progresses, fibroblasts become the primary cells at the wound site. The remodeling phase begins when the collagen production starts to decline and remains balanced with its breakdown rate. The quality and quantity of the matrix deposited during this healing phase can have a significant effect on the scar strength. In various embodiments, a nanofiber network can fully exert similar physiological and biochemical reactions to those exerted by natural ECM. Furthermore, in certain embodiments, multiple therapeutic agents can be simultaneously incorporated into the nanofiber structure to increase the functionality and enhance the regenerative capacity of the nanofiber structure.

Previous systems are mostly made of pure fibers and studies have actually struggled to succeed in removing beads and/or defects to produce homogeneous, long, continuous fiber systems with adequate mechanical properties and all other needed properties depending on ultimate application. When blends of materials are used, or even coaxial systems, the expected result is as mentioned, a system consisting of long, continuous, and homogeneous fiber. In some cases, fiber systems undergo post processing, such as annealing, to effectively remove residual solvents and/or alter the surface of the fiber to increase surface area within the long, continuous fibers. As mentioned above, blends of polymers and/or fillers are used, for example to add silver nanoparticles within solutions and ultimately fibers to provide the needed membranes with effective therapeutic/antimicrobial effects.

The above-described long, continuous fiber systems may significantly benefit by the development of multilayered multi-structure 3D (three-dimensional) systems to synergistically take advantage of each material and effectively control its pharmacokinetics (e.g., in the case of drug delivery and wound care). The opportunity to develop selective structured heterogeneous systems in situ presents an attractive process that has multiple advantages. The present inventors have developed 3D nanofiber based architectures to promote the development of multifunctional membranes. These 3D nanofiber based architectures may be developed in situ as one step processing utilizing a centrifugal spinning process (e.g., the Forcespinning® method). In various embodiments, bioactive, multifunctional 3D heterogeneous nanofiber systems are described herein. As used herein, “fibers” represent a class of materials that are continuous filaments or that are in discrete elongated pieces, similar to lengths of thread. Embodiments of fibers disclosed herein include a class of materials that exhibit an aspect ratio of at least 100 or higher. The term “microfiber” refers to fibers that have a minimum diameter in the range of 10 microns to 700 nanometers, in the range of 5 microns to 800 nanometers, or in the range of 1 micron to 700 nanometers. The term “nanofiber” refers to fibers that have a minimum diameter in the range of 500 nanometers to 1 nanometer, in the range of 250 nanometers to 10 nanometers, or in the range of 100 nanometers to 20 nanometers.

Embodiments described herein refer to the use of organic ingredients with complementary biological properties. For example, binary, ternary, or quaternary systems, where the fiber layer would be a polysaccharide based polymer system such as pullulan or chitosan, or a thermosensitive polymer such as Poloxamer (PLX), or a pH-sensitive polymer such as Eudragit, or biocompatible/biodegradable polyesters such as Poly(D,L-lactic acid) (PDLLA) and Poly(lactic-co-glycolic acid) (PLGA). The therapeutic agents may include a mixture of hydrophilic and hydrophobic agents to create different architectures through emulsion-based centrifugal spinning processes. The materials that are utilized as active ingredients selectively deposited and confined within the nanofiber layers may include:
Salvianolic Acids (SA);
Oleanolic Acid (OA);
Oxymatrine (OM);
Matrine (MA);
Trehalose (TH); and
Baicalin (BA).

Nanofiber based architectures containing mixtures of the above mentioned molecules may effectively complement each other’s biology to provide healing and restore lives of people suffering from wounds caused by, for example, autoimmune diseases such as psoriasis or diabetes. These molecules have previously been studied and used as powders, gels, or hydrogels but not as active ingredients confined as pure filled spheres or confined in a nanofiber environment.

In various instances, functional wound dressings with tailored physicochemical and biological properties are vital for effective treatment. The opportunity to separate the therapeutic agents provides higher interaction between growth factors and their target site while taking advantage of the above mentioned gains when using nanofiber scaffolds. The binary to quaternary systems can also be highly effective when dissimilar needs are present such as the need of active therapeutics and antimicrobial agents to isolate wounds from infection. Having the 3D system (multiple layered system) provides the release of the bioactive molecules or drugs while effectively maintaining the “gauze” like coverage, thus protecting the wound from further damage, and enhancing the regenerative capacity of the system.

In various embodiments, the porosity, pore size, size, and number of spheres containing the active ingredients and several other factors can be adjusted through processing parameters depending on the ultimate needs. These factors will affect microorganism penetration, oxygen permeability, pharmacokinetics, and water vapor transmission (to maintain ideal moisture condition for gas exchange). The high surface area of nanofibers is favorable for drug loading and sustained delivery and handling of the smart multifunctional wound dressing or drug delivery system. Besides nanofiber based membranes developed via a centrifugal spinning process providing the ability to effectively heal wounds (given the ability for cells to migrate through the different layers as the fiber size has larger average diameter and standard deviation when compared with other fiber spinning methods such as electrospinning), the larger standard deviation provides a variation in pore size that has proven effective in generating cell growth and proliferation for deep wounds and for hemostatic applications. Accordingly, the making of centrifugally spun 3D heterogeneous fine fiber based membranes using active ingredients and fiber substrates may be highly beneficial to different fields, particularly wound care and drug delivery.

The use of molecules such as the above mentioned molecule has been demonstrated in the development of nanoparticles. It is well known that the interaction of nanoparticles with its environment is largely governed by the properties of its outermost surface layer. Trehalose has shown the ability to provide unique biological stabilization effects when polymerized. The poly(trehalose), as well as trehalose, has the property to lower the phase transition energy associated with the freeze-drying process, protecting the biological function of the encapsulated active component. For example, the poly(trehalose) may protect siRNA polyplexes during freeze-drying and allow the nanoparticles resuspension without affecting its biological function. Given the opportunity to develop the 3D nanofiber based architectures, the need to polymerize trehalose can be avoided and a facile system to effectively utilize these molecules may result by, as mentioned above, providing the encapsulating and confining it within the nanofiber layers. Recent developments in polymerizing trehalose may also provide the advantage to use poly(trehalose) as the fiber system itself and therefore effectively carrying any of the other mentioned bioactive molecules.

Previous studies have focused on the use of the molecules mentioned above (and combinations thereof) for cancer management as alternatives or, in some cases, only solutions given adverse reactions, drug resistance, or inadequate target specificity of single anti-cancer agents. Cancer treatment often leads to chemoresistance. Some of these natural compounds and its mixtures have shown the ability to provide multiple specific targets with minimal acceptable side-effects and are therefore of wide interest to researchers due to their cytotoxic and chemo sensitizing activities. The opportunity to have these molecules selectively confined within a nanofiber scaffold provides the ability for in situ treatment such as implantable meshes, bone/skin regeneration, and ability to provide transdermal drug delivery through free standing membranes. Various embodiments are described herein that detail information for the above mentioned molecules and their effectiveness in cancer therapy, wound care, and drug delivery.

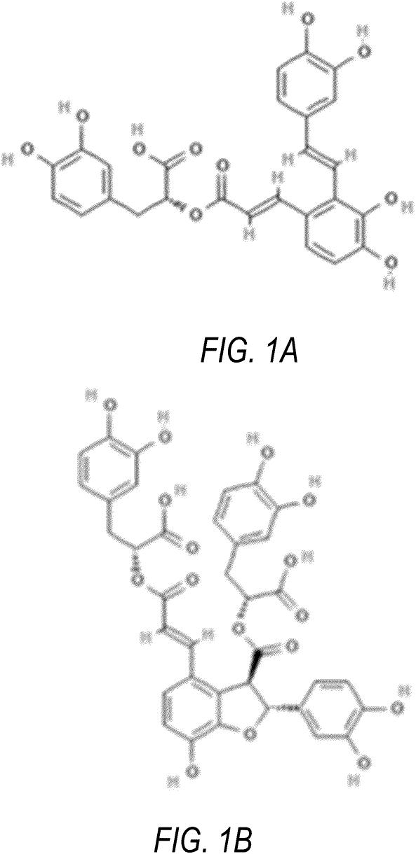

Salvianolic acids (e.g., C₂₆H₂₂O₁₀-C₃₆H₃₀O₁₆) are water soluble compounds extracted from salvia miltiorrhiza (Danshen) and are well known for their good anti-oxidative activity. Additionally, salvia miltiorrhiza, which can promote blood circulation and relieve congestion, is widely used in patients suffering from cardiovascular diseases, hyperlipidemia, and acute ischemic stroke. There are more than 10 different salvianolic acids; however, the most abundant are Salvianolic acid A (shown in FIG. 1A) and Salvianolic acid B (shown in FIG. 1B), where the Danshensu [(R)-3- (3, 4-Dihydroxyphenyl)-2- hydroxypropanoic acid] is the basic chemical structure. Recently, more studies have demonstrated that salvianolic acids may also have a good effect on the alleviation of fibrosis disease, especially on liver and pulmonary fibrosis, and the treatment of cancer.

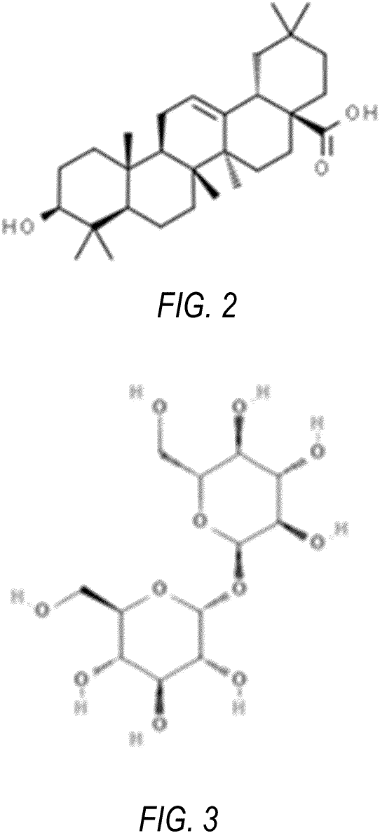

Oleanolic acid (OA) (C₃₀H₄₈) and its derivatives have been isolated from several food and medicinal plants. OA is a pentacyclic triterpenoid compound (shown in FIG. 2) abundant in plants of the Oleaceae family such as the olive plant. The OA exert promising pharmacological actions including anti-inflammatory, neuroprotective, hepatoprotective, anti-osteoporotic, anti-microbial, anti-tumor, and antidiabetics at low doses. There is some evidence that shows that OA and its derivatives have crucial prophylactic and therapeutic potential for diseases including ulcerative colitis, multiple sclerosis, metabolic disorders, diabetes, hepatitis, cardiovascular diseases, and different cancers.

Trehalose (TH) (C₁₂H₂₂O₁₁) is a nonreducing disaccharide assembled from two glucose units linked in a glycosidic linkage (shown in FIG. 3). Trehalose is biosynthesized in many organisms including bacteria, yeast, fungi, insects, invertebrates, and lower and higher plants, but not in humans; however, the enzyme trehalase is expressed in humans, which promotes metabolism into glucose. TH is a safe neuroprotective agent used in preclinical and clinical studies. Several studies have validated trehalose’s neuroprotective mechanism of action in multiple neurodegenerative diseases. An effective action in vivo is produced with a high concentration of trehalose. This molecule has high hydrophilicity and due to the trehalase enzymes (produce by cells in the small intestine), which catalyzes the conversion of trehalose to glucose, affecting its oral absorption. Assembly of trehalose (and all others) in emulsions to encase the bioactive payload and confining these within a nanofiber membrane may provide the advantage to release the free drug at its site of action, when a biologically labile linkage is used.

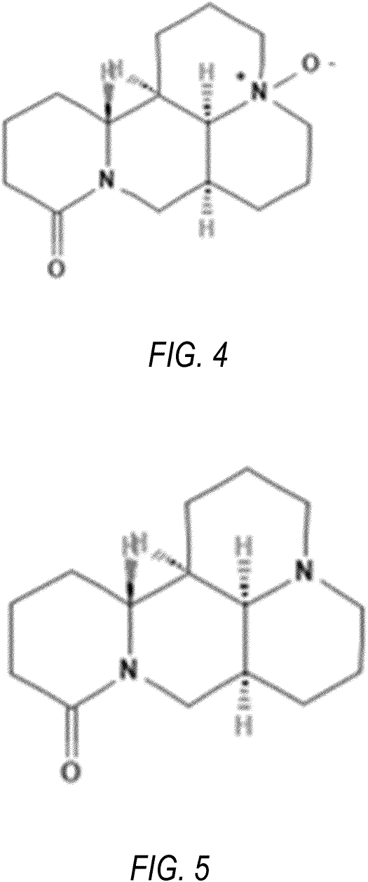

Oxymatrine (OM) (C₁₅H₂₄N₂O₂) is a pharmacologically useful natural quinolizidine alkaloid (shown in FIG. 4) extracted from the roots of the Sophora flavescent. OM has found important applications in the attenuation of chronic inflammation that promote tissue fibrosis. OM has also found use as an anti-tumor agent in experimental tumor models, suggesting broad applications in biology. In vitro and in vivo studies have demonstrated that oxymatrine exerts anti-cancer effects on most solid tumors. The mechanisms involved are related with the induction of cell cycle arrest and apoptosis and inhibition of activity, proliferation, invasion, migration, and angiogenesis. OM has a strong effect with lethal dose of around 300 mg/kg and up to 14 weeks of administrations. Oxymatrine is also a potent inhibitor of epidermal cell proliferation that is the hallmark of severe plaque psoriasis.

In some instances, Oxymatrine exerts organ- and tissue-protective effects by regulating inflammation, oxidative stress, apoptosis, and fibrosis.

### Anti-Inflammatory Effect

The inflammation process is a response triggered by damage to living tissues, in which a series of cellular signals are activated or released. Nuclear factor NF-κB consists of a family of transcription factors that has a critical role in the inflammation process. Nuclear factor NF-κB can be activated by a variety of stimulators and it participates in regulating the expression of immune-related genes. Oxymatrine may reduce the activation of the NF-κB pathway, mainly by increasing the inhibitor of NF-κB (IκB) proteins and decreasing NF-κB-p65 and phosphorylation-IκB (pIκB). The oxymatrine anti-pruritic and anti-inflammatory efficacy in the imiquimod-induced psoriasis mice was studied in vivo. In vivo studies confirmed that heat shock protein (HSP) 90 and 60 take part in the inhibitory effect of oxymatrine on the phenotypes of psoriasis mice. In vitro studies have shown that oxymatrine inhibits the expression of HSP90 and HSP60 in keratinocytes through MAPK signaling pathway; resulting in relief for psoriasis pruritic and inflammation.

### Anti-Oxidative Stress

The organs and tissues produce highly active molecules such as reactive oxygen species (ROS), which can produce a strong oxidation environment and lead to serious damage to the organs or tissues. Based on studies, the oxidative stress could increase for the malondialdehyde (MDA) presence. There are oxidant enzymes, such as catalase (CAT), glutathione peroxidase (GSH-Px), and superoxide dismutase (SOD), that play a protective role for organs and tissues in an oxidative environment. Oxymatrine could decrease the production of ROS and MDA, and could promote the levels of CAT, GSH-Px, and SOD as well.

### Anti-Apoptotic and Pro-Apoptotic Effects

Apoptosis is an important mechanism to keep the stability and homeostasis of the internal environment. Apoptosis can be divided into two main pathways: 1) the extrinsic pathway is produced by the cell surface death receptor such as fatty acid synthase (Fas)/Fas ligand (FasL), and the intrinsic pathway, which can be activated by cytotoxic stimuli and regulated by the B-cell lymphoma 2 (Bcl-2) family proteins. In most disease models, oxymatrine could protect damaged organs and tissues by reducing apoptosis through increasing the Bcl-2.

### Anti-Fibrotic Effect

Fibrosis is a pathological process in which an excessive deposition of connective tissue is caused by chronic repair after tissue and organ damage. Continuous progress of fibrosis causes significant damage to the structure and function of the organs and tissues. Fibrotic tissue often produce organ malfunction and is commonly associated with high morbidity and mortality. The transforming growth factor beta (TGF-β) is one of the strongest pro-fibrotic cytokines and the smads (group of related intracellular proteins) act as its receptors with signal transduction ability. In previous studies, the oxymatrine effect has been reported in decreasing the expression of TGF-β and smad, resulting in anti-fibrotic effect. Additionally, studies have revealed that Oxymatrine could inhibit collagen production, reduce Myeloperoxidase (MPO) activity, and decrease Hydroxyproline (Hyp) and Malondialdehyde (MDA) content.

### Anti-Cancer Effect

Oxymatrine has potential anti-cancer effects associated with two mechanisms by inducing cell cycle arrest to inhibit cell proliferation or inducing pro-apoptotic effect on tumor cells. Oxymatrine regulates various oncogenic signaling pathways to exert its cytotoxicity against cancer cells. Oxymatrine treatment has shown a decrease in tumor cell viability in various cancer cell types such as bladder, breast, cervical, colorectal, gallbladder carcinoma, gastric, osteosarcoma, among others. However, the cytotoxicity of Oxymatrine is minimal on non-cancerous cell lines such as normal lung fibroblast cells, normal bronchial epithelial cells, keratinocytes, as well as normal prostate epithelial cells.

Matrine (C₁₅H₂₄N₂O) is a quinolizidine alkaloid extracted from Sophora Flavescens; traditional Chinese herb medicines (shown in FIG. 5). Matrine, as well as OM, may be used to treat various diseases due to its potent anti-viral and anti-inflammatory effects. In previous studies, matrine was reported to reduce cellular growth and invasion potential of castration-resistant prostate cancer (CRPC) cell via suppression of matrix metalloproteases (MMP)-9 and MMP-2 activities. Additionally, it has been reported that matrine inhibits the growth of several organ-specific cancers such as breast cancer, gastric cancer, gallbladder cancer, osteosarcoma, and hepatocellular carcinoma by modulating pro-survival cell signaling pathways and the induction of apoptosis.

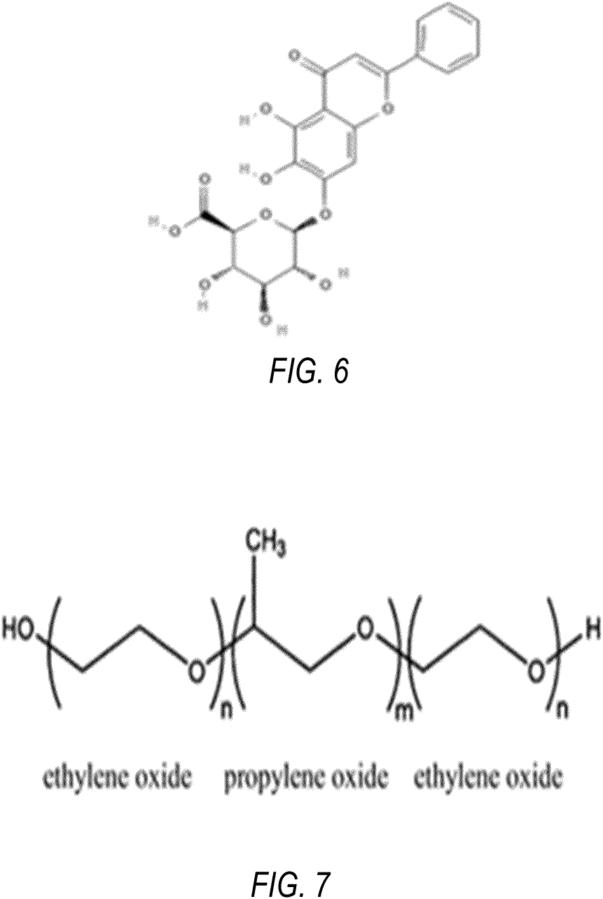

Baicalin (C₂₁H₁₈O₁₁) is a flavone glycoside widely distributed in plants of the genus Scutellaria (S. lateriflora and S. galericulata) (shown in FIG. 6). Baicalin has an extraordinary anti-inflammatory potential that can be used for the treatment of rheumatoid arthritis, respiratory diseases, inflammatory bowel diseases, cardiovascular diseases, hepatitis, kidney diseases, and neurodegenerative diseases. Baicalin may have an anti-inflammatory effect and attenuate pulmonary hypertension through the regulation of the p38 MAPK signaling pathway. Baicalin has also shown inhibited dermal inflammation and irritation in a murine model of psoriasis via topical application.

As described herein, various embodiments of the nanofiber layers could also include thermosensitive polymers such as Poloxamer, pH-sensitive such as Eudragit, etc.

Poloxamer (PEO-PPO-PEO) is a thermosensitive non-ionic block copolymer comprised by hydrophilic blocks of poly (ethylene oxide) and hydrophobic blocks of poly (propylene oxide)(shown in FIG. 7). Poloxamer has attractive properties for being used in drug delivery systems - such as non-toxicity, stable, well tunable lower critical gelation temperature (LCGT), flexible and versatility in use. In aqueous solution, due to the amphiphilic properties of the poloxamer, a block segregation may occur that promotes the formation of interesting nanostructures, which are spontaneously formed in solution (e.g., by self-assembly). The self-assembly micelles are produced when the copolymer concentration reaches the critical micellar concentration (CMC). In the micelles the PPO segments form the core and the PEO segments form the external shell. When the temperature increases, the micelles can change the structure and with a copolymer concentration between 20% and 40%, a face-centered cubic structure can be produced. This copolymer has been used in different applications such as drug delivery, nanoparticle synthesis, cosmetics and emulsion formulation, effective dispersants for inks/pigments, and as versatile anti-biofouling coatings. For example, the combination of poloxamer/ chitosan/ sodio hyaluronate as mucoadhesive in-situ gel for clonazepam (CLZ) has been shown to produce an effective gelation time, mucoadhesion time, and drug release properties by intranasal delivery. For fiber formation, several reports have shown hydrophilic poloxamer-based nanofibers developed as a novel drug delivery system for carvedilol, scaffold for ligament tissue engineering made of poly(lactide)/poloxamer or poly(lactide)/poloxamine multiblock copolymers, fiber made of poloxamer/polyacrylonitrile (PAN) applicable for medical or biotechnological filters, among others.

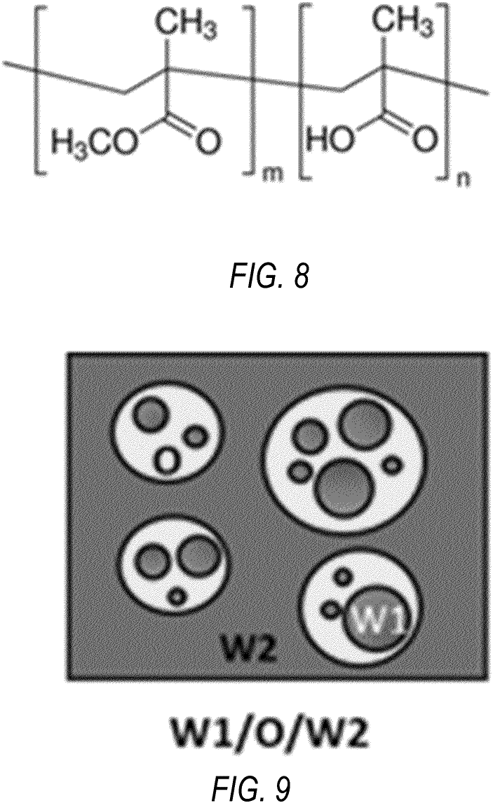

Eudragit® is a brand of a wide range of anionic, cationic and non-ionic polymethacrylate-based copolymers. These are made of methacrylic/acrylic esters or their derivatives (shown in FIG. 8). Eudragits® are non-biodegradable, nonabsorbable, and nontoxic amorphous polymers. Eudragit® is commercialized with different acidic and alkaline end groups, allowing a pH-dependent release of the active agent. For example, there are different Eudragit® formulations to be used in specific targets. Eudragit® L100-55 dissolves at a pH > 5.5 (duodenum targeting); Eudragit® L100 dissolves at a pH 6-7 (jejunum); and Eudragit® S100 at a pH > 7 (ileum and colon). The Eudragit® RL and Eudragit® SL are insoluble at physiological pH and able to swell to form permeable structure useful for drug delivery. These two Eudragit® RL and RS 100 have been selected to develop matrix films of piroxicam transdermal patches. Electrospun Eudragit® nanofibers have been produced to load different types of actives agents such as inclusion complex (IC) of niclosamide (NIC) and hydroxypropyl-beta-cyclodextrin (HPβCD) (NIC-HPβCD-IC) for anticancer treatment. Other drugs loaded on Eudragit® fiber matrix have been: 5-fluorouracil, tetracycline, budesonide, spironolactone, nifedipine, indomethacin, lecithin diclofenac sodium, paracetamol, among others.

Embodiments disclosed herein describe 3D multifunctional nanofiber based systems (see, e.g., FIG. 10 that depicts example electron microscope scans of types of 3D multifunctional nanofiber structures obtained using the procedures described herein) developed in situ using a centrifugal spinning process (e.g., the Forcespinning® technology). Various embodiments disclose 3D architectures that contain bioactive compounds such as oxymatrine/matrine/trehalose, etc. and a mixture of these as active ingredients. The present inventors have recognized that the formation of a 3D multilayered multi-structure system may synergistically provide effective use and control of the pharmacokinetics of each system. For example, the pharmacokinetics of each system may be effectively used and controlled in the case of drug delivery and wound care. The disclosed 3D bioactive, multifunctional nanofiber systems may include active ingredients encapsulated within droplets that have tunable wall thickness to control over erosion and release. The droplets may be effectively trapped/confined/secured in nanofiber layers to improve their handling. The 3D bioactive, multifunctional nanofiber systems may be developed through complex emulsions subjected to high strain rate. The physicochemical properties of the interfacially active system may prevent droplet coalescence and differentiates among fiber and droplets. The charge, density, and thickness of a layer may be affected by the properties and dimensions of the emulsifier molecules. For wound care, the making of the disclosed 3D heterogeneous systems through the Forcespinning® technique (e.g., centrifugal spinning) provides opportunity for effective cell interlayered migration (given optimal variation in fiber diameter and therefore pore size which positively affects cell growth and interlayer proliferation) to effectively heal a wound.

In various embodiments, 3D fiber structures are prepared in a 3-stage process: stage 1) solution preparation; stage 2) emulsion preparation; and stage 3) fiber production. In certain embodiments, stage 1 — solution preparation — includes the preparation of various solutions implemented in formation of the 3D fiber structures. Solutions that may be prepared include, but are not limited to, a water-soluble active components solution, a non-water-soluble active components solution, a PLGA (poly(lactic-co-glycolic acid) solution, a PEO (poly(ethylene oxide)) solution, a Poloxamer solution, and a Eudragit® solution. The following provide examples of these various solutions.

Example water-soluble active components solutions

| | | |
|---|---|---|
| | | Concentration |
| Polymer: | Chitosan | 3-5 wt.% |
| Solvent: | Deionized water | - |
| Additional components | Citric acid | 5-7 wt.% |
| Active component (AC): | Salvianolic Acids (SA) | |
| | Oxymatrine (OM) | |
| | Matrine (MA) | |
| | Trehalose (TH) | |

A water-soluble active components solution may be produced by first, preparing the acid aqueous solution by combining the water with the citric acid (CA). Once the CA is completely dissolved, the specific among of active components (AC) may be added and left under constant agitation at room temperature until complete dissolution. After complete dissolution, a polymer (e.g., chitosan) is incorporated and vortexed until a homogenous solution is obtained.

Example non-water-soluble active components solutions

| | | |
|---|---|---|
| | | Concentration |
| Polymer: | Poly(D,L-lactic acid) (PDLLA) | 5-10 wt.% |
| Solvent: | Dichloromethane or chloroform | - |
| Active component (AC): | Oleanolic Acid (OA) Baicalin (BA) | |

A non-water-soluble active components solution may be produced by first combining the solvent with the active component (AC) and leaving it under constant agitation at room temperature until complete dissolution. After complete dissolution, a polymer (e.g., PDLLA) is added and left in constant agitation until a homogenous solution is obtained.

Example PLGA solution

| | | |
|---|---|---|
| | | Concentration |
| Polymer: | Poly(lactic-co-glycolic acid) (PLGA) | 4-8 wt.% |
| Solvent: | Dichloromethane or chloroform | - |
| Surfactant | Span 80 | 0.5-2 wt.% |

A PLGA solution may be produced by first preparing a micellar solution with the solvent and span-80. Once the micellar solution is homogeneous, the polymer (e.g., PLGA) is added and the solution is left under magnetic stirring.

Example PEO solution

| | | |
|---|---|---|
| | | Concentration |
| Polymer: | Poly(ethylene oxide) (PEO)/poloxamer | 4-8 wt.% |
| Solvent: | Deionized water | - |
| Surfactant | Poloxamer | Above critical micellar concentration (CMC), and this depend on the type of poloxamer. |

A PEO solution may be produced by first preparing a micellar solution with the solvent and poloxamer. Once the micellar solution is homogeneous, the polymer (e.g., PEO/poloxamer) is added and the solution is left under magnetic stirring until complete polymer dissolution.

Example Poloxamer solution

| | | |
|---|---|---|
| | | Concentration |
| Polymer: | Poloxamer (PM) | 4-10 wt.% |
| Solvent: | Deionized water | - |
| Surfactant | - | - |

A poloxamer solution may be produced by blending the deionized water with poloxamer until a homogeneous solution is obtained.

Example Eudragit® solution

| | | |
|---|---|---|
| | | Concentration |
| Polymer: | Eudragit (Eu) | 12-20 wt.% |
| Solvent: | Deionized water | - |
| Surfactant | Tween 80 | 0.5-.2 wt.% |

A Eudragit® solution may be produced by preparing a micellar solution with the tween 80. Once the aqueous solution is homogeneous, the pH may be adjusted by adding NaOH solution until a value around 7-8 is obtained. After that, the Eudragit® is added corresponding with the concentration and left at room temperature until complete polymer dissolution.

In certain embodiments, stage 2 — emulsion preparation — includes combining various the prepared solutions to produce one-active-component emulsion systems. These emulsion systems may be combined to produce a multi-active-components emulsion system. The multi-active-components emulsion system may then be used as a final precursor emulsion system to produce 3D fiber structures with desired properties. In some embodiments, the number of active components for a final 3D fiber structure determines the number of one-active-component emulsion systems combined for obtaining the final precursor emulsion system to be use for fiber production in stage 3.

Example Emulsion Formulation and Parameters
Internal phase (IP): 10-30 v/v %.
Surfactant (External Phase (EP)): established in the solution preparation description.
Homogenization time: between 3 and 6 min.
Homogenization speed: 5,000-35,000 rpm.

In some embodiments, a multi-active-components emulsion system is used as a final precursor emulsion to produce 3D multifunctional nanofiber structures made of water-soluble active components solutions. In such embodiments, four chitosan solutions may be prepared with each of the four active components for water-soluble active components described above. The chitosan solution is the internal phase (IP) while a prepared PLGA solution is the external phase (EP). In containers (e.g., small glass vials), the PLGA solution may be added first and then the Chitosan solution corresponding to the w/o emulsion ratio established (for example, if we want 20 v/v% IP and the total amount of emulsion is 5 mL, the EP would be 4 mL and IP would be 1 mL). The system is then homogenized for a predetermined time period (e.g., 3-6 min). The same may be done for each chitosan solution corresponding to each active component. In a final step, the four emulsions, one-active-component emulsion systems, may be mixed in equal proportions or in a proportion required based on the target purpose based on the application..

In some embodiments, a multi-active-components precursor emulsion system is used as a final precursor emulsion to produce 3D multifunctional nanofiber structures made of non-water-soluble active components solutions. In such embodiments, two PDLLA solutions with each of the active components is prepared. In this instance, the PDLLA solution is the internal phase (IP) and the external phase (EP) is the PEO, poloxamer (PM), or Eudragit® (Eu) solution. In containers (e.g., small glass vials), the external phase is added first and then the internal phase is added corresponding to the w/o emulsion ratio established (for example, if we want 20 v/v % IP and the total amount of emulsion is 5 mL, the EP would be 4 mL and IP would be 1 mL). The system is homogenized for the predetermined (established) time period (e.g., 3-6 min). The same process may be done using each of the PDLLA solutions corresponding to each active component. In a final step, the two emulsions may be mixed (carefully) in equal proportions or in a proportion required based on the target purpose for the application.

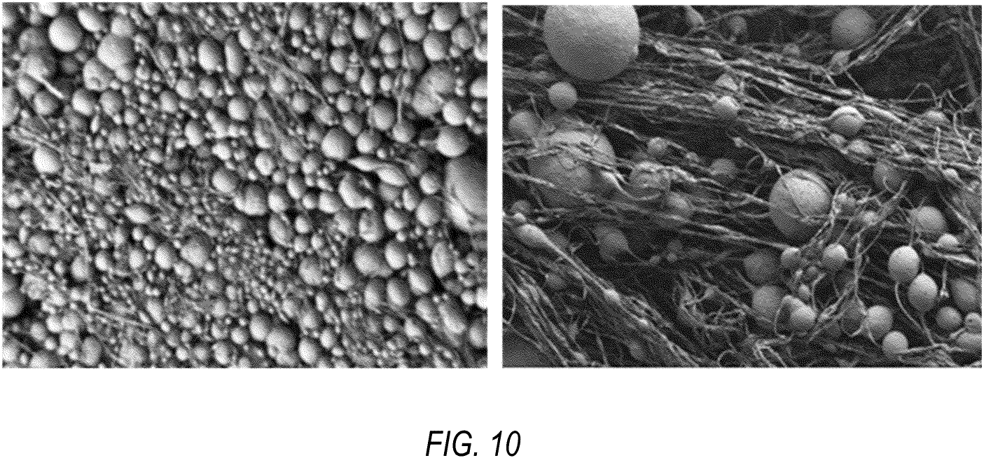

In some embodiments, a multi-active-components precursor emulsion system is used as a final precursor emulsion to produce 3D multifunctional nanofiber structures made of both water-soluble active components solutions and non-water-soluble active components solutions. In such embodiments, a multiplex emulsion (W₁/O/W₂) (shown in FIG. 9) is prepared. In certain embodiments, W₁ is the chitosan solutions with the different water-soluble active components, O is the PDLLA solution with the different non-water-soluble active components, and W₂ is PEO, PM, or Eu solutions.

In various embodiments, the combination may be of two water soluble AC with two non-water-soluble AC and, accordingly, two W₁/O and W₁/O/W₂ are prepared. The W₁/O emulsion may be prepared using the same procedures described above. For example, in the containers (e.g., vials), the PDLLA solution may be added first with OA and then the chitosan solution with OM. The resulting blend may be homogenized in order to get one of the W₁/O emulsions. The same may be done with the other combinations (e.g., maybe BA with SA). After that, the resulting emulsion may be added dropwise into a PEO, PM, or Eu solution with constant agitation (e.g., between 2000-5000 rpm) and using the corresponding internal phase proportion (10-30 v/v %). To produce the multi-active-components emulsion system, final precursor emulsion system, the two emulsions (W₁/O/W₂) may be (carefully) mixed in equal proportions or in a proportion required based on the target purpose of each one.

After a desired final precursor emulsion system is prepared, in certain embodiments, stage 3 — fiber production — is used to prepare the 3D fiber structures with desired properties. In various embodiments, the final precursor emulsion system is provided to a centrifugal spinning process (e.g., Forcespinning®). The centrifugal spinning process may be either a batch process or a continuous process. Angular velocities in the centrifugal spinning process may vary between about 2-20 k depending on the desired 3D heterogeneous fiber based membrane structure (e.g., desired droplet size/shape and fiber size).

### Example Method

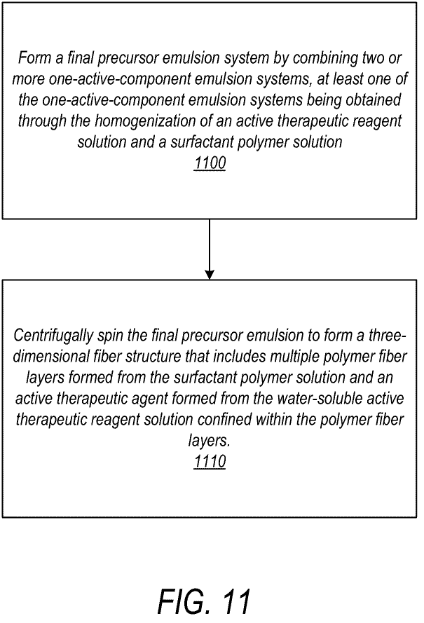

FIG. 11 is a flow diagram illustrating a method for forming a three-dimensional fiber structure, according to some embodiments. In various embodiments, some of the method elements shown may be performed concurrently, in a different order than shown, or may be omitted. Additional method elements may also be performed as desired.

At 1100, in the illustrated embodiment, a final precursor emulsion system is formed by combining two or more one-active-component emulsion systems, at least one of the one-active-component emulsion systems being obtained through the homogenization of an active therapeutic reagent solution and a surfactant polymer solution.

At 1110, in the illustrated embodiment, the final precursor emulsion is centrifugally spun to form a three-dimensional fiber structure that includes multiple polymer fiber layers formed from the surfactant polymer solution and an active therapeutic agent formed from the water-soluble active therapeutic reagent solution confined within the polymer fiber layers.

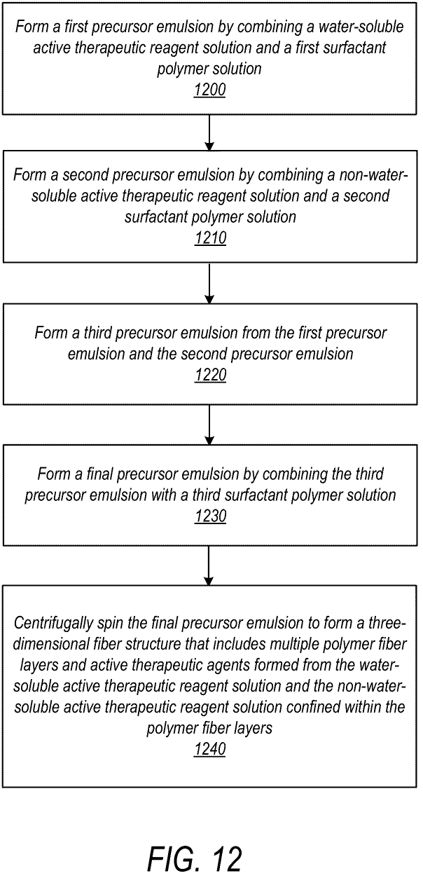

FIG. 12 is a flow diagram illustrating another method for forming a three-dimensional fiber structure, according to some embodiments. In various embodiments, some of the method elements shown may be performed concurrently, in a different order than shown, or may be omitted. Additional method elements may also be performed as desired.

At 1200, in the illustrated embodiment, a first precursor emulsion is formed by combining a water-soluble active therapeutic reagent solution and a first surfactant polymer solution.

At 1210, in the illustrated embodiment, a second precursor emulsion is formed by combining a non-water-soluble active therapeutic reagent solution and a second surfactant polymer solution.

At 1220, in the illustrated embodiment, a third precursor emulsion is formed from the first precursor emulsion and the second precursor emulsion.

At 1230, in the illustrated embodiment, a final precursor emulsion is formed by combining the third precursor emulsion with a third surfactant polymer solution.

At 1240, in the illustrated embodiment, the final precursor emulsion is centrifugally spun to form a three-dimensional fiber structure that includes multiple polymer fiber layers and active therapeutic agents formed from the water-soluble active therapeutic reagent solution and the non-water-soluble active therapeutic reagent solution confined within the polymer fiber layers.

The present disclosure includes references to “an “embodiment” or groups of “embodiments” (e.g., “some embodiments” or “various embodiments”). Embodiments are different implementations or instances of the disclosed concepts. References to “an embodiment,” “one embodiment,” “a particular embodiment,” and the like do not necessarily refer to the same embodiment. A large number of possible embodiments are contemplated, including those specifically disclosed, as well as modifications or alternatives that fall within the spirit or scope of the disclosure.

This disclosure may discuss potential advantages that may arise from the disclosed embodiments. Not all implementations of these embodiments will necessarily manifest any or all of the potential advantages. Whether an advantage is realized for a particular implementation depends on many factors, some of which are outside the scope of this disclosure. In fact, there are a number of reasons why an implementation that falls within the scope of the claims might not exhibit some or all of any disclosed advantages. For example, a particular implementation might include other circuitry outside the scope of the disclosure that, in conjunction with one of the disclosed embodiments, negates or diminishes one or more the disclosed advantages. Furthermore, suboptimal design execution of a particular implementation (e.g., implementation techniques or tools) could also negate or diminish disclosed advantages. Even assuming a skilled implementation, realization of advantages may still depend upon other factors such as the environmental circumstances in which the implementation is deployed. For example, inputs supplied to a particular implementation may prevent one or more problems addressed in this disclosure from arising on a particular occasion, with the result that the benefit of its solution may not be realized. Given the existence of possible factors external to this disclosure, it is expressly intended that any potential advantages described herein are not to be construed as claim limitations that must be met to demonstrate infringement. Rather, identification of such potential advantages is intended to illustrate the type(s) of improvement available to designers having the benefit of this disclosure. That such advantages are described permissively (e.g., stating that a particular advantage “may arise”) is not intended to convey doubt about whether such advantages can in fact be realized, but rather to recognize the technical reality that realization of such advantages often depends on additional factors.

Unless stated otherwise, embodiments are non-limiting. That is, the disclosed embodiments are not intended to limit the scope of claims that are drafted based on this disclosure, even where only a single example is described with respect to a particular feature. The disclosed embodiments are intended to be illustrative rather than restrictive, absent any statements in the disclosure to the contrary. The application is thus intended to permit claims covering disclosed embodiments, as well as such alternatives, modifications, and equivalents that would be apparent to a person skilled in the art having the benefit of this disclosure.

For example, features in this application may be combined in any suitable manner. Accordingly, new claims may be formulated during prosecution of this application (or an application claiming priority thereto) to any such combination of features. In particular, with reference to the appended claims, features from dependent claims may be combined with those of other dependent claims where appropriate, including claims that depend from other independent claims. Similarly, features from respective independent claims may be combined where appropriate.

Accordingly, while the appended dependent claims may be drafted such that each depends on a single other claim, additional dependencies are also contemplated. Any combinations of features in the dependent that are consistent with this disclosure are contemplated and may be claimed in this or another application. In short, combinations are not limited to those specifically enumerated in the appended claims.

Where appropriate, it is also contemplated that claims drafted in one format or statutory type (e.g., apparatus) are intended to support corresponding claims of another format or statutory type (e.g., method).

What is claimed is:

1. A fiber system, comprising:
a three-dimensional fiber structure comprising multiple polymer fiber layers, wherein the fiber layers are formed from two or more polymers;
an active therapeutic agent confined in the polymer fiber layers;
wherein the three-dimensional fiber structure has been formed by centrifugal spinning of a final precursor emulsion containing the two or more polymers and the active therapeutic agent.

2. The system of claim 1, wherein the active therapeutic agent is one of the following: salvianolic acid, oleanolic acid, oxymatrine, matrine, trehalose, and baicalin.

3. The system of claim 1, wherein the active therapeutic agent is hydrophilic.

4. The system of claim 1, wherein the active therapeutic agent is hydrophobic.

5. The system of claim 1, wherein the three-dimensional fiber structure includes two or more active therapeutic agents.

6. The system of claim 1, wherein at least one of the polymers is a polysaccharide-based polymer, and wherein the polysaccharide-based polymer is pullulan or chitosan.

7. The system of claim 1, wherein at least one of the polymers is a thermosensitive polymer.

8. The system of claim 7, wherein the thermosensitive polymer includes at least one of the following polymers: poloxamer, a pH-sensitive polymer, a biocompatible polymer, a biodegradable polyester, poly(D,L-lactic acid) (PDLLA), and poly(lactic-co-glycolic acid) (PLGA).

9. The system of claim 1, wherein the active therapeutic agent is confined within the fiber layers in the three-dimensional fiber structure.

10. The system of claim 1, wherein the active therapeutic agent is encapsulated within droplets in the three-dimensional fiber structure, and wherein a wall thickness of the droplets is tuned to provide a controlled release rate for the active therapeutic agent.

11. A method, comprising:
forming a final precursor emulsion system by combining two or more one-active-component emulsion systems, at least one of the one-active-component emulsion systems being obtained through the homogenization of an active therapeutic reagent solution and a surfactant polymer solution; and
centrifugally spinning the final precursor emulsion to form a three-dimensional fiber structure that includes multiple polymer fiber layers formed from the surfactant polymer solution and an active therapeutic agent formed from the water-soluble active therapeutic reagent solution confined within the polymer fiber layers.

12. The method of claim 11, further comprising:
forming a second precursor emulsion by combining a second active therapeutic reagent solution and a second surfactant polymer solution;
combining the second precursor emulsion with the precursor emulsion in a predetermined ratio to form a final precursor emulsion; and
centrifugally spinning the final precursor emulsion to form the three-dimensional fiber structure, wherein the three-dimensional fiber structure includes the active therapeutic agent formed from the active therapeutic reagent solution and a second active therapeutic agent formed from the second active therapeutic reagent solution confined within the polymer fiber layers.

13. The method of claim 12, wherein the active therapeutic reagent solution is a water-soluble active therapeutic reagent solution, and wherein the second active therapeutic reagent solution is a non-water-soluble active therapeutic reagent solution.

14. The method of claim 11, wherein the active therapeutic reagent solution is a water-soluble active therapeutic reagent solution, the water-soluble active therapeutic reagent solution including one of the following active therapeutic agents: salvianolic acid, oleanolic acid, oxymatrine, and matrine.

15. The method of claim 14, wherein the water-soluble active therapeutic reagent solution includes a polysaccharide-based polymer.

16. The method of claim 15, wherein the multiple polymer fiber layers are formed from the surfactant polymer solution and the polysaccharide-based polymer.

17. The method of claim 11, wherein the active therapeutic reagent solution is a non-water-soluble active therapeutic reagent solution, the non-water-soluble active therapeutic reagent solution including one of the following active therapeutic agents: trehalose and baicalin.

18. The method of claim 17, wherein the water-soluble active therapeutic reagent solution includes a thermosensitive polymer, and wherein the multiple polymer fiber layers are formed from the surfactant polymer solution and the thermosensitive polymer.

19. The method of claim 11, wherein the surfactant polymer solution includes a thermosensitive polymer.

20. A method, comprising:
forming a first precursor emulsion by combining a water-soluble active therapeutic reagent solution and a first surfactant polymer solution;
forming a second precursor emulsion by combining a non-water-soluble active therapeutic reagent solution and a second surfactant polymer solution;
forming a third precursor emulsion from the first precursor emulsion and the second precursor emulsion;
forming a final precursor emulsion by combining the third precursor emulsion with a third surfactant polymer solution; and
centrifugally spinning the final precursor emulsion to form a three-dimensional fiber structure that includes multiple polymer fiber layers and active therapeutic agents formed from the water-soluble active therapeutic reagent solution and the non-water-soluble active therapeutic reagent solution confined within the polymer fiber layers.
